SEQUENCE LISTING (2 sequences)

SEQ ID NO: 1 - protein, 414 aa, Artificial Sequence
  synthetic polypeptide
GDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPAKRKKKGGKNGKNRRNRKKKNP

SEQ ID NO: 2 - protein, 415 aa, Artificial Sequence
  synthetic polypeptide
MGDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPAKRKKKGGKNGKNRRNRKKKNP

(19) United States
(12) Patent Application Publication
(10) Pub. No.: US 2014/0342006 A1
(43) Pub. Date: Nov. 20, 2014

(54) Methods for Preparing Injectable Protein Microparticle Suspensions

(71) Applicant: Ansun Biopharma, Inc., San Diego, CA (US)

(72) Inventor: Tiejun Li, San Diego, CA (US)

(73) Assignee: Ansun Biopharma, Inc., San Diego, CA (US)

(21) Appl. No.: 14/209,350

(22) Filed: Mar. 13, 2014

Related U.S. Application Data
(60) Provisional application No. 61/800,484, filed on Mar. 15, 2013.

(51) Int. Cl. A61K 9/16; A61K 38/47

(52) U.S. Cl. CPC A61K 9/1641; A61K 38/47
USPC 424/499; 424/94.61

(57) ABSTRACT

A method for preparing a microparticle suspension, which comprises: (a) providing protein microparticles having a median diameter between 5 and 13 microns and a GSD less than 2.5; (b) combining the microparticles with a liquid, pharmaceutically acceptable, water miscible media to create a first mixture; (c) adding an aqueous media to the first mixture to create second mixture; and (d) mixing the second mixture to create a microparticle suspension is described. The suspension are injectable even when they contain a relatively high concentration of protein.

### RELATED APPLICATION

This application claims priority to U.S. Provisional Application Ser. No. 61/800,484, filed Mar. 15, 2013, the entire contents are which hereby incorporated by reference.

### BACKGROUND

In order to be optimally therapeutically effective certain proteins, e.g., antibodies, need to be administered at a relatively high dose and are preferably administered by subcutaneous injection.

Due to the relatively low volume of subcutaneous injection, often less than 2 ml per injection, a subcutaneously injected therapeutic protein is preferably present at a relatively high concentration. However, some proteins, for example, monoclonal antibodies, exhibit concentration-driven protein aggregation at high concentrations which can lead to poor stability and high viscosity. High viscosity negatively impacts injectability.

### SUMMARY

Described herein are methods for creating injectable suspensions of proteins, and in particular injectable suspensions of protein-containing microparticles.

The methods entail providing protein-containing microparticles having a median diameter (on a volume or weight basis) of 5-15 microns, in some cases 7 to 14, 8-13 or 8-11 microns, and have a narrow range of size distribution (geometric standard deviation (“GSD”) less than 3 (or less than 2), e.g., between 1.5 and 2.5. First, a water-miscible media is added to the microparticles. Once the water-miscible media is thoroughly mixed with the microparticles, an aqueous solution is added to the mixture to create a suspension of the microparticles.

In one embodiment, the method for preparing a microparticle suspension, comprises: (a) providing protein microparticles having a median diameter between 5 and 13 microns and a GSD less than 2.5; (b) combining the microparticles with a liquid, pharmaceutically acceptable, water miscible media to create a first mixture; (c) adding an aqueous media to the first mixture to create second mixture; and (d) mixing the second mixture to create a microparticle suspension.

In another embodiments, the method for preparing microparticle suspension consists essentially of: (a) providing protein microparticles having a median diameter between 5 and 13 microns and a GSD less than 2.5; (b) combining the microparticles with a liquid, pharmaceutically acceptable, water miscible media to create a first mixture; (c) adding an aqueous media to the first mixture to create second mixture; and (d) mixing the second mixture to create a microparticle suspension.

In various embodiments of the forgoing methods: the volume of the liquid, pharmaceutically acceptable, water miscible media added is equal to or greater than the volume of aqueous media added; the protein concentration of suspension is greater than 10 mg/ml, greater than 50 mg/ml or greater than 100 mg/ml; or between 10 mg/ml and 100 or 200 mg/ml; the liquid, pharmaceutically acceptable, water miscible media is selected from: polyethylene glycol, polysorbate, propylene glycol, thioglycerol, tricaprylin, triolein, and versetamide; the method is carried out between 5 and 30° C.; consists of (or comprises) water and a pharmaceutically acceptable salt; the liquid, pharmaceutically acceptable, water miscible media consists of: polyethylene glycol, polysorbate, propylene glycol, thioglycerol, tricaprylin, triolein, or versetamide and a pharmaceutically acceptable salt; and protein microparticles are at least 25%, 50% or 75% w/w protein; the water miscible media comprises: polyethylene glycol, polysorbate 80, polysorbate 20 (Polyoxyethylene (20) sorbitan monooleate), propylene glycol, thioglycerol, tricaprylin, triolein, and versetamide; the ratio of water miscible media added to aqueous media added is between 35:65 and 65:35 on a volume basis; the microparticles comprise DAS181; and the microparticle concentration in the suspension is 0.01-0.5 mg/ml; and the microparticle concentration in the suspension is 0.01-0.2 mg/ml.

Also described herein is a microparticle suspension made by any of the forging methods.

### DETAILED DESCRIPTION

Initial efforts examined suspending microparticles in various oils (e.g., safflower oil) and in water an aqueous media media (e.g., polyethylene glycol, Tween, ETOCA, Pluronic, Chremophor). Two aspects of the suspensions were studied: viscosity and injectability (force required for injection through a needle). It was found that in some cases the viscosity of the microparticles suspended in oil increased exponentially with the concentration of the microparticles, while the force required for injection rose approximately linearly with the increase in particle concentration.

Aqueous media were also examined because they can be very stable, are expected to have little impact on the PK/PD of the protein, and many are pharmaceutically acceptable. Moreover, certain aqueous media used in pharmaceutical applications are highly purified using, for example, a flash chromatography process that can remove polar impurities, such as peroxide species, aldehydes and ketones. The removal of such impurities from the media eliminates their adverse interactions with APIs, and improves stability. However, aqueous media can cause gelling, increased viscosity and can solubilize the protein microparticles. In the testing with protein microparticle, many of the aqueous media dissolved the microparticles.

After extensive testing, a two step method for producing suspension of protein microparticles was developed. The method entails: 1) mixing the protein microparticles with a pharmaceutically acceptable, water miscible media; and then 2) adding water, optionally containing salts or buffers, to the mixture of protein microparticles and pharmaceutically acceptable, water miscible media.

### Production of Protein Microparticles

Methods for producing suitable protein microparticles are described in PCT/US2007/001914. Briefly, the protein microparticle preparation includes the steps of mixing together a solution of a protein, e.g., an antibody, in an aqueous solvent, a counterion and a solvent (e.g., isopropanol) and cooling the resulting mixture (also referred to herein as cocktail solution or feedstock solution) to a predetermined temperature below about 25° C. at a cooling rate that is maintained at a constant fixed value until the mixture is at a predetermined temperature below about 25° C.

In many cases there are two or more cooling phases during which the temperate is decreased at a fixed rate. In some cases the solution is held for a period of time at a predetermined temperature. The resulting microparticles can be separated from the mixture to remove components other than the microparticles by, for example, sedimentation, filtration and/or freeze-drying.

The size of the microparticles of the resulting formulations is controlled, in large part, by a combination of the choice of counterion and the cooling rate. In general, the faster the cooling rate, the smaller the size of the resulting microparticles. The uniformity of the size is achieved in certain cases by maintaining the cooling rate at a constant, fixed value until the mixture is cooled to the desired predetermined temperature below about 25° C. Thus, the cooling rate is maintained regardless of the changing temperature differential during cooling, i.e., the difference between the temperature of the cocktail solution at any given time during the cooling process and the final predetermined temperature to which it is cooled.
Counterion

The selection and characterization of counterions has been described extensively elsewhere and is incorporated by reference herein (US 20070190163 and US 2010/0166874). Suitable counterions include: amin acids (e.g., histidine, magnesium sulphate and citric acid and citrate).
Nature and Concentration of Organic Solvent

An organic solvent added to the cocktail in the methods provided herein generally is not a polymer, generally can be water miscible and is selected from among alcohols described in US20070190163 US 20100166874 A1. In some embodiments of the methods provided herein, the organic solvent is isopropanol. In general, the organic solvent isopropanol is a good solvent of choice because (1) it is a class 3 solvent (i.e., safe), (2) it can produce microspheres in a wide range (2-30%, v/v) of concentrations, and (3) it has a relatively high freezing point so its vapors can efficiently be trapped during lyophilization. In particular embodiments of the methods provided herein, the final concentration of isopropanol is 25% or 26%.
Cooling Ramp

The feedstock solutions from which microparticles are formed according to the methods provided herein are cooled at a constant, fixed preset rate—beginning at a temperature of above or at 25° C. at which the feedstock solution initially is present, and ending at a predetermined temperature below about 25° C. at which the microparticles are formed. The predetermined temperature at which microparticles are formed is empirically determined based on the type of macromolecule, solvents, counterions and other ingredients as well as the rate of cooling and can vary from about or at 15° C., 10° C., 8° C., 5° C., 3° C., 2° C., 1° C., —2° C., —5° C., —7.5° C., —10° C., −15° C., —20° C., −25° C., −30° C., −35° C., −40° C., −45° C., −50° C. or −55° C.

The rate at which cooling and freezing of the cocktail (cooling ramp) is performed can determine the final size of the microparticles. In general, a faster cooling ramp yields smaller microparticles whereas a slower cooling ramp yields larger microparticles. Depending on the size of microparticles desired and the type of active agent, the cooling rate can be from about 0.01° C./min to about 1° C./min. In general, the cooling rate is less than 1° C./min and is about 0.3, 0.4, 0.5, 0.6, 0.7 or 0.8° C./min.

### Production of DAS181 Microparticles

Purification of DAS 181

DAS 181 is a fusion protein containing the heparin (glysosaminoglycan, or GAG) binding domain from human amphiregulin fused via its N-terminus to the C-terminus of a catalytic domain of Actinomyces Viscosus (e.g., sequence of amino acids set forth in SEQ ID NO: 1 (no amino terminal methionine) and SEQ ID NO: 2 (including amino terminal methionine). The DAS 181 protein used in the examples below was purified as described in Malakhov et al., Antimicrob. Agents Chemother., 1470-1479 (2006), which is incorporated in its entirety by reference herein. Briefly, the DNA fragment coding for DAS 181 was cloned into the plasmid vector pTrc99a (Pharmacia) under the control of an IPTG (isopropyl-β-D-thiogalactopyranoside)-inducible promoter. The resulting construct was expressed in the BL21 strain of Escherichia Coli (E. Coli). The E. coli cells expressing the DAS181 protein were washed by diafiltration in a fermentation harvest wash step using Toyopearl buffer 1, UFP-500-E55 hollow fiber cartridge (GE Healthcare) and a Watson-Marlow peristaltic pump. The recombinant DAS 181 protein was then purified in bulk from the cells as described in US 20050004020 and US 20080075708, which are incorporated in their entirety by reference herein.
Activity of DAS 181

The sialidase activity of DAS181 was measured using the fluorogenic substrate 4-methylumbelliferyl-N-acetyl-α-D-neuraminic acid (4-MU-NANA; Sigma). One unit of sialidase is defined as the amount of enzyme that releases 10 nmol of MU from 4-MU-NANA in 10 minutes at 37° C. (50 mM CH₃COOH—NaOH buffer, pH 5.5) in a reaction that contains 20 nmol of 4-MU-NANA in a 0.2 ml volume (Potier et al., Anal. Biochem., 94:287-296, 1979). The specific activity of DAS181 was determined to be 1,300 U/mg protein (0.77 μg DAS181 protein per unit of activity).
Microparticle Preparation

The following ingredients were then combined to form DAS181 microparticles in a large scale batch process:
(a) 75 mg/ml Histidine, 0.107M citric acid, pH 5.0 and 1M Trehalose stock solutions were sterile filtered into and combined in an Excipient Bottle.
(b) The contents of the Excipient Bottle were added, with mixing, to a Compounding Vessel containing 125 mg/ml DAS181 protein prepared as described in Example 1.
(c) Isopropanol was sterile filtered into an Isopropanol Bag
(d) The content of the Isopropanol Bag was pumped into the Compounding Vessel while mixing vigorously to form the Feedstock Solution. The final composition of the Feedstock Solution was as follows: 70 mg/ml DAS181, 26% isopropanol, 9.8 mg/ml histidine, 9.8 mg/ml trehalose, 2.69 mg/ml citric acid, pH 5.0. The time between initiating the addition of isopropanol and starting the lyophilization cycle was between 90 minutes and 120 minutes
(e) Stainless Steel trays that had undergone depyrogenation were each filled with 950 g of the Feedstock Solution, using a metering pump
(f) The filled Stainless Steel trays were subjected to a Lyophilization Cycle as follows:
   a. the feedstock solution in the lyophilization trays were gasketed and placed in the lyophilizer shelves at 25° C. for 5 minutes;
   b. the temperature of the shelves was lowered to −55° C. at a ramp rate of −0.4° C./minute;
   c. the trays were held at −55° C. for between 60 and 180 minutes;
   d. primary drying was accomplished by setting the condenser to <−60° C., applying a vacuum of 125 mTorr with 250 mTorr dead band and increasing the temperature to −40° C. at a ramp rate of 0.125° C./minute and further to a temperature of −30° C. at 0.167° C./minute;
   e. the temperature was held at −30° C. for between 5000 and 6500 minutes;
   f. secondary drying was accomplished by increasing the temperature to 15° C. at a ramp rate of 0.5° C./minute, holding at 15° C. for 30 minutes, then further ramping up to a temperature of 30° C. at a ramp rate of 0.5° C./minute;
   g. the temperature was held at 30° C. for between 300 and 500 minutes; and
   h. the vacuum was released and the lyophilizer was backfilled with nitrogen to prevent oxidation of the microparticle formulations before transferring into bottles for bulk mixing and aliquoting the bulk powder for storage at ≦−15° C.
Physical Parameters:

The DAS 181 dry powder microparticles prepared according to the above method have a mass median aerodynamic diameter (MMAD) of about 10 microns and a GSD of between 1 and 2.
DAS181 Sequences

DAS 181 (without amino terminal Met)
(SEQ ID NO: 1)
GDHPQATPAPAPDASTELPASMSQAQHLAANTATDNYRIPAITTAPNGDLLISYDERPKDNGNG
GSDAPNPNHIVQRRSTDGGKTWSAPTYIHQGTETGKKVGYSDPSYVVDHQTGTIFNFHVKSYDQ
GWGGSRGGTDPENRGIIQAEVSTSTDNGWTWTHRTITADITKDKPWTARFAASGQGIQIQHGPH
AGRLVQQYTIRTAGGAVQAVSVYSDDHGKTWQAGTPIGTGMDENKVVELSDGSLMLNSRASDGS
GFRKVAHSTDGGQTWSEPVSDKNLPDSVDNAQIIRAFPNAAPDDPRAKVLLLSHSPNPRPWSRD
RGTISMSCDDGASWTTSKVFHEPFVGYTTIAVQSDGSIGLLSEDAHNGADYGGIWYRNFTMNWL
GEQCGQKPAKRKKKGGKNGKNRRNRKKKNP
DAS 181 (with amino terminal Met)
(SEQ ID NO: 2)
MGDHPQATPAPAPDASTELPASMSQAQHLAANTATDNYRIPAITTAPNGDLLISYDERPKDNGN
GGSDAPNPNHIVQRRSTDGGKTWSAPTYIHQGTETGKKVGYSDPSYVVDHQTGTIFNFHVKSYD
QGWGGSRGGTDPENRGIIQAEVSTSTDNGWTWTHRTITADITKDKPWTARFAASGQGIQIQHGP
HAGRLVQQYTIRTAGGAVQAVSVYSDDHGKTWQAGTPIGTGMDENKVVELSDGSLMLNSRASDG
SGFRKVAHSTDGGQTWSEPVSDKNLPDSVDNAQIIRAFPNAAPDDPRAKVLLLSHSPNPRPWSR
DRGTISMSCDDGASWTTSKVFHEPFVGYTTIAVQSDGSIGLLSEDAHNGADYGGIWYRNFTMNW
LGEQCGQKPAKRKKKGGKNGKNRRNRKKKNP

### Suspension of Microparticles

To prepare 1 ml of a 100 mg DAS181/ml suspension, 125 mg of microparticles prepared as described were placed in a vial in a controlled RH environment (typically 10-30% RH). Next, 450 μL of PEG 300 was added to the vial and gently mixed with the microparticles. The mixture was held for 5 minutes to allow the microparticles to interact with the PEG 300. Next, 450 μL of water is added to the vial and the contents are gently mixed for 2-3 minutes or until a homogeneous suspension is achieved.

Injectability was measured using a NE-1010 syringe pump with a DPM-3 digital mount meter attached to the plunger rail. Standard 1 mL BD syringes are used with 27G×½ PrecisionGlide BD needles. Injectability values are reported in unit of lbs of force measured. Viscosity was measured using a Brookfield DV-1 Prime with a CPE-44PY cup and a CPE-40 cone spindle. Injection force of less then 50N is considered as injectable. The conversion unit of lbs to N is 1 lbs=4.4 N.

The above method produced suspensions with good injectability. Good results were obtained when the ratio of PEG 300 to water was: 50:50, 65:35 and 75:25. When PEG 200 was used, good results were obtained when the ratio of PEG 300 to water was 65:35 and 75:25.

In addition to polyethylene glycol (PEG 200, PEG 300, PEG 400, PEG 500, PEG 600), polysorbate 80, polysorbate 20 (Polyoxyethylene (20) sorbitan monooleate), propylene glycol, thioglycerol, tricaprylin, triolein, and versetamide are useful first media for adding to the protein microparticles.

The second media is water that can include salts, buffers, preservatives and other pharmaceutically acceptable excipients.

What is claimed is:

1. A method for preparing a microparticle suspension, comprising:
(a) providing protein microparticles having a median diameter between 5 and 13 microns and a GSD less than 2.5;
(b) combining the microparticles with a liquid, pharmaceutically acceptable, water miscible media to create a first mixture;
(c) adding an aqueous media to the first mixture to create second mixture; and
(d) mixing the second mixture to create a microparticle suspension.

2. The method of claim 1 wherein the volume of the liquid, pharmaceutically acceptable, water miscible media added is equal to or greater than the volume of aqueous media added.

3. The method of claim 1 wherein the protein concentration or microparticle concentration of suspension is greater than 10 mg/ml, greater than 50 mg/ml or greater than 100 mg/ml and less than 500 mg/ml.

4. The method of claim 1 wherein liquid, pharmaceutically acceptable, water miscible media is selected from: polyethylene glycol, polysorbate, propylene glycol, thioglycerol, tricaprylin, triolein, and versetamide.

5. The method of claim 1 wherein the method is carried out between 5 and 30° C.

6. The method of claim 1 wherein the aqueous media consists of water and a pharmaceutically acceptable salt.

7. The method of claim 1 wherein liquid, pharmaceutically acceptable, water miscible media consists of: polyethylene glycol, polysorbate, propylene glycol, thioglycerol, tricaprylin, triolein, or versetamide and a pharmaceutically acceptable salt.

8. The method of claim 1 wherein the protein microparticles are at least 25%, 50% or 75% w/w protein.

9. A method for preparing a microparticle suspension, consisting essentially of:
(a) providing protein microparticles having a median diameter between 5 and 13 microns and a GSD less than 2.5;
(b) combining the microparticles with a liquid, pharmaceutically acceptable, water miscible media to create a first mixture;
(c) adding an aqueous media to the first mixture to create second mixture; and
(d) mixing the second mixture to create a microparticle suspension.

10. A suspension of protein microparticles produced by the method of claim 1 or the method of claim 9.

11. The method of claim 1 or claim 9 wherein the water miscible media comprises:
polyethylene glycol, polysorbate 80, polysorbate 20 (Polyoxyethylene (20) sorbitan monooleate), propylene glycol, thioglycerol, tricaprylin, triolein, and versetamide are useful first media for adding to the protein microparticles.

12. The method of claim 1, 9, or 11 wherein the ratio of water miscible media added to aqueous media added is between 35:65 and 65:35 on a volume basis.

13. The method of claim 1 or claim 9 whereien the microparticles comprise DAS181.

14. The method of claim 1 or claim 9 wherein the microparticle concentration in the suspension is 0.01-0.5 mg/ml.

15. The method of claim 1 or claim 9 wherein the microparticle concentration in the suspension is 0.01-0.2 mg/ml.

16. A microparticle suspension produced by any of claims 1, 9 and 13.

17. Then method of claim 1 or claim 9 wherein the suspension is has an injection force of less than 50N when injected using 22-27 gauge needle that is 0.5-1.5 inches long.
